(19) United States
(12) Patent Application Publication
(10) Pub. No.: US 2003/0026844 A1
(43) Pub. Date: Feb. 6, 2003

(54) Injectable sustained release pharmaceutical composition and processes for preparing the same

(76) Inventors: Hee-Yong Lee, Iksan-shi (KR); Hye-suk Lee, Iksan-shi (KR); Jung-Soo Kim, Jeonju-shi (KR); Sang-Beom Kim, Kunsan-shi (KR); Ji-Suk Lee, Kunsan-shi (KR); Ho-Il Choi, Taejon (KR); Seung-Gu Chang, Tajeon (KR)

Correspondence Address: Eric B Meyertons, Conley, Rose, & Tayon, P.C., P O Box 398, Austin, TX, 78767, US

(21) Appl. No.: 10/018,870

(22) PCT Filed: Mar. 22, 2001
(86) PCT No.: PCT/KR01/00462

(30) Foreign Application Priority Data
Apr. 18, 2000 (KR) 2000/20484
Aug. 24, 2000 (KR) 2000/49344

(51) Int. Cl. A61K 9/16; A61K 9/50

(52) U.S. Cl. 424/493; 424/499; 424/501

(57) ABSTRACT

The present invention relates to controlled and sustained release pharmaceutical composition and processes for preparing the same. The present invention is to provide processes to prepare an injectable sustained release pharmaceutical composition comprising a step to prepare biodegradable porous microspheres having accessible ionic functional groups, a step to incorporate a biopharmaceutical into the microspheres through ionic interaction by suspending or equilibrating the microspheres in a solution containing the biopharmaceutical and a step to recover and freeze-dry the biopharmaceutical-incorporated microspheres.

### FIELD OF THE INVENTION

The present invention relates to controlled and sustained release pharmaceutical composition and processes for preparing the same.

### BACKGROUND ART

Recent advances in recombinant biotechnology have resulted in a proliferation of new biopharmaceutical such as proteins and peptides. However, most proteins and peptides have poor oral absorption and very short half-lives after being administered by parenteral routes such as intravenous, subcutaneous and intramuscular injections. As a consequence, repetitive injection, infusion or sustained release dosage forms are required to obtain a desired therapeutic efficacy in a patient.

For the purpose of obtaining in vivo sustained release of therapeutic proteins and peptides for a prolonged time, biodegradable natural and synthetic polymeric materials have been extensively studied for the carriers [Heller, J. et al., Controlled release of water-soluble macromolecules from bioerodible hydrogels, Biomaterials, 4, 262-266 (1983); Langer, R., New methods of drug delivery, Science, 249, 1527-1533 (1990); Okada, H. and Toguchi, H., Biodegradable microspheres in drug delivery, Crit. Rev. Ther. Drug Carrier Syst., 12, 1-99 (1995)]. Among the biodegradable polymers, aliphatic polyesters including polylactides, polyglycolides and their copolymers have been mostly investigated due to the great biocompatibility and variable time range of biodegradability dependent on their physical properties such as co-monomer ratio, molecular weight and hydrophilicity [DeLuca, P. P. et al., Biodegradable polyesters for drug and polypeptide delivery, in: El-Nokaly, M. A., Piatt, D. M., and Charpentier, B. A. (Eds.), Polymeric delivery systems, properties and applications, American Chemical Society, pp. 53-79 (1993); Park, T. G., Degradation of poly(lactic-co-glycolic acid) microspheres: effect of copolymer composition, Biomaterials, 16, 1123-1130 (1995); Anderson, J. M. and Shive, M. S., Biodegradation and biocompatibility of PLA and PLGA microspheres, Adv. Drug. Del. Rev., 28, 5-24 (1997); Tracy, M. A. et al., Factors affecting the degradation rate of poly(lactide-co-glycolide) microspheres in vivo and in vitro, Biomaterials, 20, 1057-1062 (1999)].

Several methods including solvent extraction and evaporation, phase separation and spray drying which were used for encapsulation of conventional chemical drugs have also been used to prepare protein-loaded poly(D,L-lactide-co-glycolide) (PLGA) microspheres [McGee, J. P. et al., Zero order release of protein from poly(D,L-lactide-co-glycolide) microparticles prepared using a modified phase separation technique, J. Controlled Rel., 34, 77-86 (1995); Gander, B. et al., Quality improvement of spray-dried, protein-loaded D,L-PLA microspheres by appropriate polymer solvent selection, J. Microencapsul., 12, 83-97 (1995); O'Donnell, P. B. and McGinity, J. W., Preparation of microspheres by the solvent evaporation technique, Adv. Drug Del. Rel., 28, 25-42 (1997), U.S. Pat. Nos. 4,818,542, 5,942,253]. Due to the hydrophilic nature of most protein drugs, water in oil in water (w/o/w) double emulsion solvent evaporation technique is frequently used for encapsulating protein into a biodegradable polymeric matrix. In this process, an aqueous protein solution is emulsified into a polymer-solvent phase and this primary emulsion is further dispersed into a large volume of water phase containing an appropriate surfactant. Inevitably, protein drugs are exposed to a water/organic solvent interface. Most protein drugs are denatured and non-covalently aggregated during this primary emulsion stage. Resultantly, the final product of protein-loaded microspheres typically showed an initial burst release of relatively native protein portions which were loosely bound to polymeric microspheres followed by no significant release of irreversibly aggregated protein portions for any prolonged time [Kim, H. K. and Park, T. G., Biotechnol. Bioeng., 65, 659-667 (1999), Crotts, G. and Park, T. G., J. Microencapsul., 15, 699-713 (1998)].

Several efforts have been tried to minimize denaturation and aggregation of protein during encapsulation process. With the use of excipients such as trehalose, mannitol, dextran, heparin and polyethylene glycol in aqueous protein solution, some stabilizing effects had been obtained [U.S. Pat. No. 5,804,557, Cleland, J. L. and Jones, A. J. S., Pharm. Res., 13, 1464-1475 (1996), Cleland, J. L. et al., Pharm. Res., 14, 420-425 (1997), Pean, J. M. et al., Pharm. Res., 16, 1294-1299 (1999), Sanchez, A. et al., Int. J. Pharm., 185, 255-266 (1999), Lavelle, E. C. et al., Vaccine, 17, 516-529 (1999)]. These excipients partly seemed to prevent protein denaturation by forming a hydration layer around the protein and reducing the protein-organic solvent interactions. Using a solid protein powder instead of an aqueous protein solution is another effort to minimize exposure of protein into a water-organic solvent interface [Cleland, J. L. and Jones, A. J. S., Stable formulations of recombinant hGH and interferon-γ for microencapsulation in biodegradable microspheres, 13, 1464-1475 (1996); Iwata, M. et al., Particle size and loading efficiency of poly(D,L-lactic-co-glycolic acid) multiphase microspheres containing water soluble substances prepared by the hydrous and anhydrous solvent evaporation methods, J. Microencapsul., 16, 49-58 (1999)]. However, in this process, the protein still will encounter an aqueous environment in the presence of organic solvent during secondary emulsion step. Therefore above two approaches could not completely protect protein from denaturation and aggregation during microencapsulation procedures.

Alternative strategy to develop protein-loaded microspheres is to soak protein solution into porous PLGA microspheres [U.S. Pat. No. 5,470,582, Duggirala, S. S. et al., Pharm. Dev. Technol., 1, 11-19 (1996), Duggirala, S. S. et al., Pharm. Dev. Technol., 1, 165-174 (1996), Schrier, J. A. and DeLuca, P. P., Pharm. Dev. Technol., 4, 611-621 (1999)]. In this method, protein will never have a chance to come into a water-organic solvent interface. Although this approach successfully applied to incorporate protein drugs,such as recombinant human bone morphogenetic protein-2 into porous PLGA microspheres, loading capacity was limited to a low level, that is, 1 mg protein/g microspheres (0.1% loading).

Therefore, there is a need to provide a method for encapsulating a biopharmaceutical such as protein or peptide in its fully active state into biodegradable microspheres with high drug loading.

### DISCLOSURE OF THE INVENTION

The present invention provides injectable sustained release pharmaceutical compositions, processes for preparing the compositions. Particularly, the present invention provides a process for encapsulating a biopharmaceutical such as peptide and protein in a fully active state into biodegradable microspheres wherein the content of biopharmaceutical is significantly increased compared to prior art methods.

The process consists of three steps comprising a first step to prepare porous biodegradable polymeric microspheres containing accessible ionic functional groups, a second step to incorporate a biopharmaceutical into the ionic porous microspheres by suspending the microspheres in an aqueous solution of biopharmaceutical and a third step of recovering and freeze-drying the biopharmaceutical-loaded microspheres. According to the present invention, the incorporation of a biopharmaceutical into polymeric microspheres is mainly achieved through ionic interaction between ionic functional groups of porous polymeric microspheres and counter ionic groups of a biopharmaceutical.

Therefore, this invention has two main advantages for preparing sustained release biopharmaceutical compositions. One is a protection of the denaturation and irreversible aggregation of the biopharmaceutical during incorporation process because the incorporation is achieved under absence of an organic solvent that is very harmful to a biopharmaceutical especially under co-existence of aqueous solution. The other is a highly attainable biopharmaceutical content in the pharmaceutical composition as the incorporation capacity of the porous microspheres is drastically increased due to the introduction of ionic functional groups into the microspheres.

### BRIEF DESCRIPTION OF DRAWINGS

FIGS. 1a and 1b depict overall schematic illustrations of the incorporation mechanism of biopharmaceutical into ionic porous microspheres according to the present invention. FIG. 1a illustrates the incorporation mechanism of cationic biopharmaceuticals into anionic porous microspheres. FIG. 1b illustrates the incorporation mechanism of anionic biopharmaceuticals into cationic porous microspheres.

FIGS. 2a, 2b and 2c are optical microscopic photographs of ionic porous PLGA microspheres prepared according to this invention. FIG. 2a shows anionic porous microspheres prepared by the procedure described in Example 1, formulation-2. FIG. 2b shows cationic porous microspheres prepared by the procedure described in Example 1, formulation-7. FIG. 2c shows human growth hormone (hGH)-loaded microspheres (16.83% drug content) prepared by incorporation of hGH into the cationic microspheres shown in FIG. 2b.

FIG. 3 shows the time kinetics of lysozyme incorporation into anionic microspheres shown in FIG. 2a at two different temperatures.

FIG. 4 shows the effect of pH of incorporation medium on the lysozyme incorporation capacity of anionic microspheres shown in FIG. 2a.

FIG. 5 shows the effect of NaCl concentration of incorporation medium on the lysozyme incorporation capacity of anionic microspheres shown in FIG. 2a.

FIG. 6 shows in vitro release of hGH from hGH-loaded microspheres (16.83% drug content) shown in FIG. 2c.

### BEST MODE FOR CARRYING OUT THE INVENTION

One aspect of the present invention is to provide processes to prepare an injectable sustained release pharmaceutical composition comprising a step to prepare biodegradable porous microspheres having accessible ionic functional groups, a step to incorporate a biopharmaceutical into the microspheres through ionic interaction by suspending or equilibrating the microspheres in a solution containing the biopharmaceutical and a step to recover and freeze-dry the biopharmaceutical-incorporated microspheres.

Another aspect of the present invention is to provide said processes, wherein the composition is prepared by incorporation of a cationic biopharmaceutical into biodegradable porous microspheres having anionic functional groups and wherein the pH of incorporation solution is lower than the pI of the biopharmaceutical.

Another aspect of the present invention is to provide said processes, wherein the composition is prepared by incorporation of an anionic biopharmaceutical into biodegradable porous microspheres having cationic functional groups and wherein the pH of incorporation solution is higher than the pI of the biopharmaceutical.

Another aspect of the present invention is to provide said processes, wherein said biopharmaceutical is present in an amount from 0.1% to 90% weight.

Another aspect of the present invention is to provide said processes, wherein said biodegradable polymer is one or more of polylactides, polyglycolides, poly(lactide-co-glycolide)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and polyorthoester, biodegradable polyurethanes, proteins such as albumin, casein, collagen, fibrin, fibrinogen, gelatin, hemoglobin, transfferin, and zein, polysaccharides such as alginic acid, chitin, chitosan, chondroitin, dextrin, dextran, hyaluronic acid, heparin, keratan sulfate, starch and derivatives or blends thereof.

Another aspect of the present invention is to provide said processes, wherein said anionic functional groups are selected from carboxyl, sulfonyl and phosphoryl groups.

Another aspect of the present invention is to provide said processes, wherein said biodegradable porous microspheres having anionic functional groups are prepared from the blends of anionic surfactant and/or biocompatible materials having anionic functional group with biodegradable polymer.

Another aspect of the present invention is to provide said processes, wherein said anionic surfactant is selected from docusate sodium and sodium lauryl sulfate.

Another aspect of the present invention is to provide said processes, wherein said cationic functional groups are selected from primary to quaternary amine groups.

Another aspect of the present invention is to provide said processes, wherein said biodegradable porous microspheres having cationic functional groups are prepared from the blends of cationic surfactant or biocompatible materials having cationic functional group with biodegradable polymer.

Another aspect of the present invention is to provide said processes, wherein said cationic surfactant is selected from benzalkonium chloride, benzethonium chloride, and cetrimide.

Another aspect of the present invention is to provide said processes, wherein said biopharmaceutical is selected from the group consisting of growth hormones, interferons, colony stimulating factors, interleulins, macrophage activating factors, macrophage peptides, B cell factors, T cell factors, protein A, suppressive factor of allergy, suppressor factors, cytotoxic glycoprotein, immunocytotoxic agents, immnunotoxins, immunotherapeutic polypeptides, lymphotoxins, tumor necrosis factors, cachectin, oncostatins, tumor inhibitory factors, transforming growth factors, albumin and its fragments, alpha-1 antitrypsin, apolipoprotein-E, erythroid potentiating factors, erythropoietin, factor VII, factor VIII, factor IX, fibrinolytic agent, hemopoietin-1, kidney plasminogen activator, tissue plasminogen activator, urokinase, prourokinase, streptokinase, lipocortin, lipomodulin, macrocortin, lung surfactant protein, protein C, protein 5, C-reactive protein, renin inhibitors, collagenase inhibitors, superoxide dismutase, epidermal growth factor, platelet derived growth factor, osteogenic growth factors, atrial naturetic factor, auriculin, atriopeptin, bone morphogenetic protein, calcitonin, calcitonin precursor, calcitonin gene-related peptide, cartilage inducing factor, connective tissue activator protein, fertility hormones (follicle stimulating hormone, leutinizing hormone, human chorionic gonadotropin), growth hormone releasing factor, osteogenic protein, insulin, proinsulin, nerve growth factor, parathyroid hormone, parathyroid hormone inhibitors, relaxin, secretin, somatomedin C, insulin-like growth factors, inhibin, adrenocorticotrophic hormone, glucagons, vasoactive intestinal polypeptide, gastric inhibitory peptide, motilin, cholecystokinin, pancreatic polypeptide, gastrin releasing peptide, corticotropin releasing factor, thyroid stimulating hormone, vaccine antigens of, and anti-infective antibodies to, bacterial or viral or other infectious organisms and mutants or analogs thereof.

Another aspect of the present invention is to provide said processes, wherein said biodegradable porous microspheres having ionic functional groups are prepared by a method selected from solvent extraction or evaporation in aqueous or organic phase, phase separation, spray drying, low temperature casting and supercritical gas fluid method.

Another aspect of the present invention is to provide said processes, wherein porosity of said biodegradable porous microspheres having ionic functional groups is intended to be increased by addition of gas forming agents or salts such as sodium chloride, calcium chloride and ammonium bicarbonate during microsphere preparation process.

Another aspect of the present invention is to provide said processes, wherein said biodegradable porous microspheres having ionic functional groups are prepared by co-addition of acidifying agents such as lactic acid, glycolic acid, tartaric acid, citric acid, fumaric acid, and malic acid, alkalizing agents such as diethanolamine, monoethanolamine, potassium citrate, sodium bicarbonate, calcium carbonate, magnesium carbonate, magnesium oxide, magnesium trisilicate, sodium citrate, meglumine, and triethanolamine and salts.

Another aspect of the present invention is to provide said processes, wherein the incorporation of a biopharmaceutical into said biodegradable porous microspheres having ionic functional groups are performed in an aqueous buffer solution, where the pH of the buffer is from 3.0 to 9.0, salt concentration of the buffer is from 1 to 500 mM, incorporation temperature is from 5 to 50° C. and incorporation time is from 1 minute to 20 days.

Another aspect of the present invention is to provide said processes, wherein the salt concentration of the buffer is from 5 to 200 mM, incorporation temperature is from 30 to 42° C. and incorporation time is from 10 to 48 hours.

Another aspect of the present invention is to provide said processes, wherein the incorporation medium further comprises a release rate modifying additive or excipient or a cryoprotectant.

Another aspect of the present invention is to provide said processes, wherein the composition is further coated with one or more of gelatin, fibrin, or albumin.

Another aspect of the present invention is to provide said processes, wherein the size of the microspheres is within the range from 0.01 to 500 μm.

Another aspect of the present invention is to provide an injectable sustained release pharmaceutical composition by preparing said processes.

As described above, the process of this invention for preparing an injectable sustained release pharmaceutical composition comprises a step to prepare porous biodegradable polymeric microspheres containing accessible ionic functional groups, a step to incorporate a biopharmaceutical into the microspheres through ionic interaction and a step to recover and freeze-dry the biopharmaceutical-loaded microspheres.

As used herein, the term “biopharmaceutical” refers to a bioactive agent whose active portion is constructed by an amino acid sequence of varying length from about two amino acids to hundreds of amino acids, which are often referred to as peptides and proteins. Particularly, the process of this invention is valuable to biopharmaceuticals consisting of more than twenty amino acids and having molecular a weight of more than 2,000 because these biopharmaceuticals generally need to be maintained in their secondary, tertiary and quaternary structures as native states to exert their therapeutic activities which are prone to be destroyed during microencapsulating process by prior art methods. The active portion of a biopharmaceutical may also contain additional derivatizing groups such as sugars or lipids.

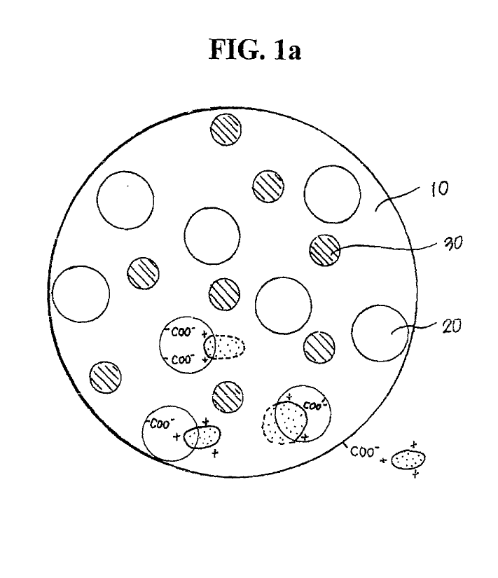

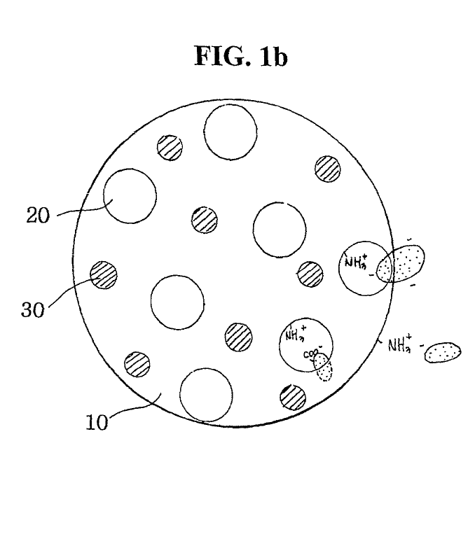

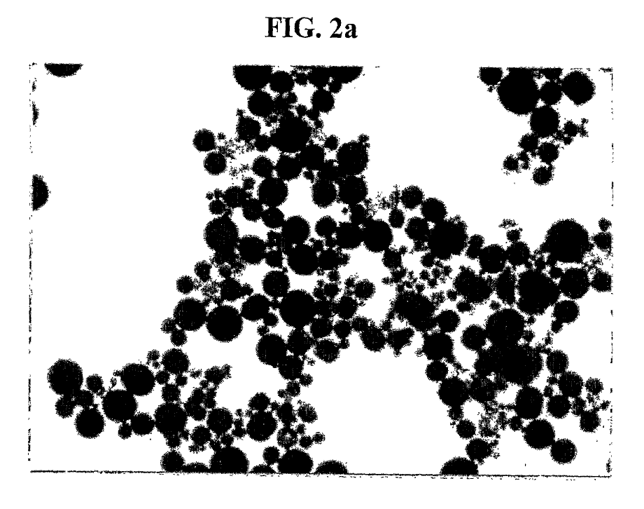

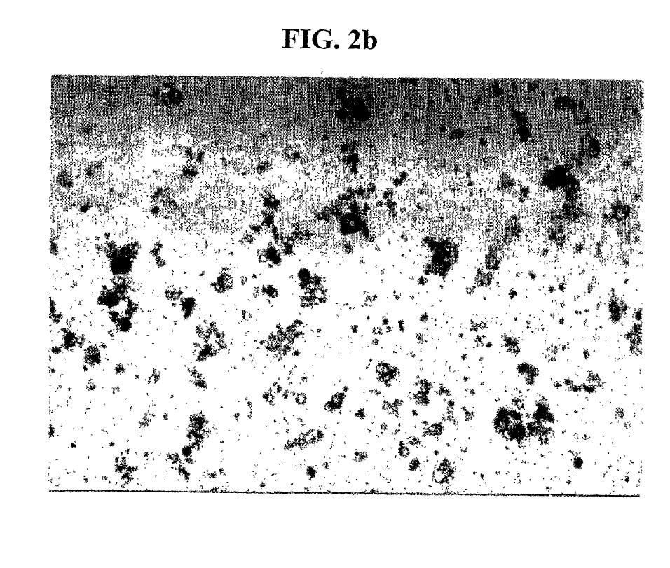

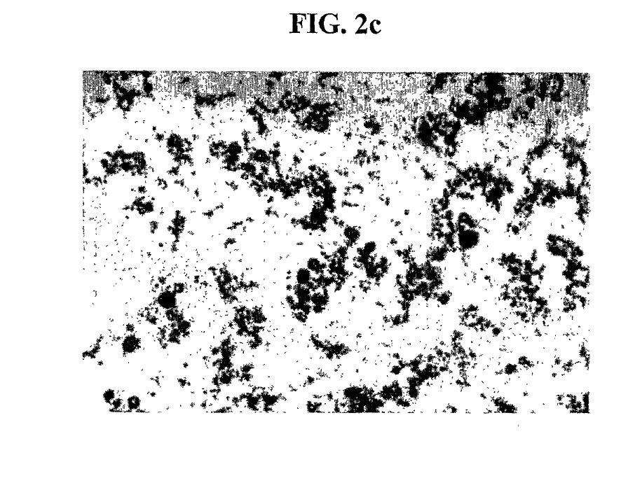

The overall incorporation mechanism, according to this invention is schematically illustrated by FIGS. 1a and 1b. FIG. 1a illustrates the incorporation mechanism of cationic biophaimaceuticals into anionic porous microsphere through ionic interaction. Anionic functional groups present on the surface and pores (20) of porous microsphere (10) are prepared from biodegradable polymers interacting with cationic groups of the biopharmaceutical.

FIG. 1b illustrates the incorporation mechanism of anionic biopharmaceutical into cationic porous microspheres through ionic interaction. Cationic functional groups present on the surface and pores of porous microspheres, are prepared from biodegradable polymers interacting with anionic groups of the biopharmaceutical. An addition of pH regulating materials (30) such as acidifying and alkalizing agents during manufacturing process of porous microspheres can regulate the biodegradation rate of the polymer and protect from an abrupt pH change in the microenvironment of microsphere resulting in a modulation of the in vivo release rate of biopharmaceutical.

Examples of polymers useful in the present invention may be found in U.S. Pat. Nos. 3,960,757, 4,818,542, 5,160,745, 5,830,493, 5,916,597, 5,942,241. In particular, preferred polymers are biodegradable polymers including synthetic polymers such as polylactides, polyglycolides, poly(lactide-co-glycolide)s, polycaprolactone, polycarbonates, polyesteramides, polyanhydrides, poly(amino acids), polyorthoesters, polyacetyls, polycyanoacrylates, polyetheresters, poly(dioxanone)s, poly(alkylene alkylate)s, copolymers of polyethylene glycol and polyorthoester, biodegradable polyurethanes and natural polymers including proteins such as albumin, casein, collagen, fibrin, fibrinogen, gelatin, hemoglobin, transfferin, and zein and polysaccharides such as alginic acid, chitin, chitosan, chondroitin, dextrin, dextran, hyaluronic acid, heparin, keratan sulfate, and starch and derivatives and/or blends thereof.

More particularly, desirable polymers are homopolymers of lactic acid, glycolic acid, or copolymers thereof, i.e., poly(lactide-co-glycolide)s. These polymers biodegrade to non-toxic monomers, lactic acid and glycolic acid and are commercially available from a number of sources. These polymers are currently used in the injectable depot formulations of therapeutic peptide and protein such as leuprorelin acetate (an agonist of luteinizing hormone-releasing hormone, Lupron Depot®) and hGH (Lutropin Depot®).

Ionic functional groups can be introduced to porous polymeric microspheres by preparing microspheres from biodegradable polymers having ionic groups or blending of biodegradable polymers not having ionic groups with biocompatible materials or biodegradable polymers having ionic groups.

Biodegradable polymers may have ionic groups intrinsically or be introduced by chemical modification methods as ordinarily defined in the art.

Examples of ionic groups intrinsically contained in biodegradable polymers include free carboxyl end groups of unblocked polyesters, carboxyl or amino groups present in poly(amino acids) and proteins, cationic or anionic groups present in polysaccharides.

Introduction of ionic groups into biodegradable polymers can be carried out by conventional chemical reactions. For example, aliphatic polyesters such as polylactides, polyglycolides, and poly(lactide-co-glycolide)s may have cationic functional groups by modification of hydroxyl or carboxyl groups therein into amino groups.

For example (Reaction I), as for the method of aminizing an amide terminal, which is a Hoffman rearrangement reaction, there is a method of first incorporating azide into the carboxyl group, followed by incorporation of alcohol, making it into amine ester, and then incorporating amine at sodium hydroxide (Tetrahedron Letters, 25, 315, 1984, Journal of Organic Chemistry, 51, 3007, 5123, 1986). By means of Lossen reaction, there is a method of converting carboxylic acid into hydroxylamine, and carboxylic acid into amine. As for the method of incorporating a diamine group into the carboxylic acid group, there is a method of activating the carboxylic acid group by means of using dicyclohexylcarbodiimide, carbonyldiimide, or Castro reagent, etc., followed by condensation reaction with a compound, such as propandiamine, butylenediamine, ehtylenediamine, or bipheyldiamine. As such, a biodegradable polymer incorporating a cationic functional group of an amine group could be obtained.

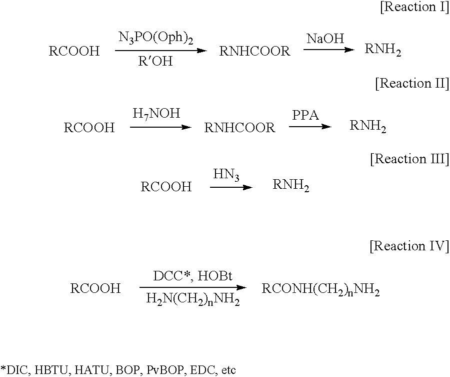

Examples of ionic groups which can be introduced into biodegradable polymers are anionic groups such as carboxyl, sulfonyl and phosphoryl groups and cationic groups such as primary to quaternary amine groups.

Examples of ionic group-containing biocompatible materials include, but are not limited to, cationic surfactants such as benzalkonium chloride, benzethonium chloride, and cetrimide and anionic surfactants such as docusate sodium and sodium lauryl sulfate, and other biocompatible materials comprising carboxyl, sulfonyl, phosphoryl or amino groups.

In vivo release rate of a biopharmaceutical may be controlled to some extent by the addition of excipients such as acidifying agents, alkalizing agents and salts during microsphere manufacturing. These excipients will be released from the matrix of microsphere during the biodegradation process and modulate the release rate of a biopharmaceutical by weakening the ionic interaction between a biopharmaceutical and an ionic group of a microsphere. Suitable excipients include, but are not limited to, acidifying agents such as lactic acid, glycolic acid, tartaric acid, citric acid, fumaric acid, and malic acid, alkalizing agents such as diethanolamine, monoethanolamine, potassium citrate, sodium bicarbonate, calcium carbonate, magnesium carbonate, magnesium oxide, magnesium trisilicate, sodium citrate, meglumine, and triethanolamine, and salts such as sodium chloride and calcium chloride.

Microspheres can be prepared by any method among ordinary prior arts, i.e., solvent extraction and/or evaporation in aqueous or organic phase, coacervation or phase separation, spray drying, low temperature casting and supercritical gas fluid method. Detailed technical aspects are well described in U.S. Pat. Nos. 3,523,906, 4,652,441, 5,288,502, 4,606,940, 5,271,961, 5,518,709, 5,019,400, and 5,043,280. Particularly, water in oil in water (w/o/w) double emulsion solvent extraction and evaporation method is preferred. In this method, fine water droplets in the primary emulsion will provide plenty of pores within a resulting solidified microsphere. The porosity of microsphere can be further increased by addition of porosigens such as sodium chloride, calcium chloride and ammonium bicarbonate. Increased surface area of microsphere due to the increased porosity may result in an increase of incorporation capacity of the microsphere toward a biopharmaceutical.

A representative list of suitable biopharmaceuticals applicable to the present invention may be found in U.S. Pat. Nos. 4,962,091, 5,288,502, 5,470,582, and 5,480,656. Of particular interests are insulin, growth hormones, prolactin, calcitonin, paratlhyroid hormone, interferons, interleukins, thymopoietin, tumor necrosis factor, colony-stimulating factors (CSFs), asparaginase, insulin-like growth factors, nerve growth factor, cell growth factors, bone morphogenetic proteins (BMPs), nerve nutrition factors, blood coagulation factors, erythropoietin, thrombopoietin, and vaccines derived from proteins of viral, bacterial and parasitic infective agents. More particular interests are hGH, erythropoietin, granulocyte-CSF, granulocyte macrophage-CSF, interferons, interleukins, BMPs and peptide or protein vaccines.

Incorporation of a biopharmaceutical into microspheres can be performed by simply suspending and/or equilibrating the microspheres in a solution having desired concentration of the biopharmaceutical. Similar procedures are described in U.S. Pat. Nos. 5,145,675 and 5,470,682. The main advance of the present invention compared to above two prior inventions is introduction of various kinds of ionic groups into the porous microspheres. Accordingly, incorporation of a biopharmaceutical of the present invention is mainly caused by ionic interaction but not by hydrophobic adsorption. Resultant advantages of the present invention are a minimal structural perturbation of biopharmaceutical caused from hydrophobic interaction with the polymer, a higher degree of biopharmaceutical incorporation and a lower initial release. Another advantage of this invention is its applicability to a broad range of biopharmaceuticals having quite different ionic characteristics by selecting a suitable kind of ionic group to be introduced into the microspheres.

When incorporating a biopharmaceutical into the ionic porous microspheres, critical considerations are the kind of ionic group within the porous microspheres, the ionic characteristic of a biopharmaceutical to be incorporated, and parameters of incorporation conditions such as pH, ionic strength, and temperature of incorporation medium and equilibration time of incorporation.

Generally, anionic microspheres having at least a group selected from carboxyl, sulfonyl, and phosphoryl groups may be used for the incorporation of a basic biopharmaceutical, i.e., of which pI is above 7.0, whereas cationic microspheres having an amino group may be used for the incorporation of an acidic biopharmaceutical, i.e., of which pI is below 7.0.

The pH of the buffer to be used as an incorporation medium can be varied from3.0to 9.0.

Salt concentration of the buffer can be varied from 1 to 500 mM, but preferably, a range from 5 to 200,mM is appropriate.

Temperature of the incorporation medium and equilibration time of incorporation should be also considered, as these factors affect hydration and swelling degrees of the microspheres, strength of the ionic interaction between a biopharmaceutical and microspheres, and stability of the biopharmaceutical. Generally, the temperature range of 0-50° C. can be used, but 37° C., a physiological temperature, is preferably used. A time period of about 1 min to about several days can be used for incorporation, but 10-48 hours is preferred in case the incorporation temperature is 37° C.

The release rate of the biopharmaceutical can be controlled to some extent by the addition of release rate modifying agents in the incorporation medium.

After incorporation has been completed, biopharmaceutical-incorporated microspheres can be separated from the incorporation medium by centrifugation or filtration and free flowing powder can be obtained by freeze-drying the microspheres.

In case the biopharmaceutical is deactivated during freeze-drying process, cryoprotectants may be added during the incorporation process or just before freeze-drying process.

Initial drug release can be further adjusted by coating of the biopharmaceutical-incorporated microspheres by gelatin, collagen, fibrin, or albumin.

The following Examples are intended to further illustrate the present invention without limiting its scope of the claims in any way.

### EXAMPLE 1

### Preparation of Porous Biodegradable Microspheres Having Ionic Functional Groups

Formulation-1: Microspheres were prepared by w/o/w double emulsion solvent evaporation method using a hydrophilic 50:50 PLGA polymer (RG502H, Boehringer Ingelheim), which contains free carboxyl end groups. Eight hundred μl of deionized water was added to 1 g of PLGA polymer dissolved in 2 ml of methylene chloride and emulsified by sonication for 30 seconds at power 1, frequency 20,000 using a probe type ultrasonic generator (Ulsso Hitech, Seoul, South Korea). This primary emulsion was dispersed into 200 ml of deionized water containing 0.5% polyvinylalcohol (w/v) in a vessel which connected to a constant temperature controller and mixed well by stirring for 15 minutes at 2,500 rpm, 25° C. using a mixer (Silverson L4RT laboratory mixer, Chesham, England). After mixing for another 15 minutes at 1,500 rpm, 25° C., temperature of continuous phase was increased to 40° C. to evaporate methylene chloride. After 1 hour stirring at 40° C., 1,500 rpm, temperature was decreased to 25° C. The hardened microspheres were collected by centrifugation and washed twice with 200 ml of deionized water, and then freeze-dried.

Formulation-2: All the manufacturing procedures are similarly performed as described in formulation-1, except 800 μl of aqueous 0.5 M NaCl instead of deionized water was used as primary water phase to increase the porosity of the microspheres.

Formulation-3: All the manufacturing procedures are similarly performed as described in formulation-1, except 800 μl of aqueous 0.5 M citric acid (pH 5.0) instead of deionized water was used as primary water phase.

Formulation-4: All the manufacturing procedures are similarly performed as described in formulation-1, except 800 μl of aqueous 0.5 M caprylic acid (pH 8.5) instead of deionized water was used as primary water phase.

Formulation-5: All the manufacturing procedures are similarly performed as described in formulation-1, except 800 μpl of aqueous 0.5 M ammonium bicarbonate (pH 7.0) instead of deionized water was used as primary water phase.

Formulation-6: All the manufacturing procedures are similarly performed as described in formulation-2, except, instead of RG502H, a hydrophobic 50:50 PLGA polymer (RG502, Boehringer Ingelheim) not having free carboxyl end groups was used as a biodegradable polymer. The stirring speed of secondary emulsion step was increased to 3,000 rpm.

Formulation-7: All the manufacturing procedures are similarly performed as described in formulations-6 with some changes as follows. In methylene chloride, 0.9 g of RG502 and 0.1 g of benzalkonium chloride, a cationic surfactant, were co-dissolved. Primary water phase was 800 μl of aqueous 0.5 M NaCl containing 40 mg of benzalkonium chloride, and secondary water phase was 200 ml of 0.5% (w/v) PVA containing 10 g of benzalkonium chloride.

Formulation-8: All the manufacturing procedures are similarly performed as described in formulation-6 with some changes as follows. In methylene chloride, 0.98 g of RG502 and 0.02 g of benzalkonium chloride were co-dissolved. Primary water phase was 800 μl of aqueous 0.5 M NaCl containing 8 mg of benzalkonium chloride and secondary water phase was 200 ml of 0.5% (w/v) PVA containing 8 g of benzalkonium chloride.

Formulation-9: All the manufacturing procedures are similarly performed as described in formulation-2, except 0.9 g of RG502H and 0.1 g of magnesium carbonate were dissolved and suspended, respectively, in methylene chloride.

Formulation-10: All the manufacturing procedures are similarly performed as described in formulation-2, except 0.9 g of RG502H and 0.1 g of magnesium hydroxide were dissolved and suspended, respectively, in methylene chloride.

Formulation-11: All the manufacturing procedures are similarly performed as described in formulation-2, except 0.95 g of RG502H and 0.05 g of benzalkonium chloride were co-dissolved in methylene chloride.

Formulation-12: All the manufacturing procedures are similarly performed as described in formulation-2, except 0.95 g of RG502H and 0.05 g of caprylic acid were co-dissolved in methylene chloride.

Formulation-13: All the manufacturing procedures are similarly performed as described in formulation-6, except 0.95 g of RG502 and 0.05 g of octylamine were co-dissolved in methylene chloride.

Formulation-14: All the manufacturing procedures are similarly performed as described in formulation-6, except 0.9 g of RG502H and 0.1 g of poly-8-CBZ-1-lysine were co-dissolved in methylene chloride.

Formulation-15: All the manufacturing procedures are similarly performed as described in formulation-6, except primary water phase was 800 μl of 0.5 M NaCl, 0.1% (w/v) chitosan, and 1% (v/v) acetic acid.

Formulation-16: All the manufacturing procedures are similarly performed as described in formulation-6, except 0.95 g of RG502 and 0.05 g of caprylic acid were co-dissolved in methylene chloride.

Formulation-17: All the manufacturing procedures are similarly performed as described in formulation-6, except 0.98 g of RG502 was dissolved in methylene chloride and primary water phase was 800 μl of 0.5 M NaCl containing 20 mg of poly(l-lysine).

### EXAMPLE 2

### Incorporation of Protein Drugs into the Microspheres

Protein drugs were incorporated through ionic interaction into the microspheres obtained from the Example 1 by simply soaking and equilibrating the microspheres into a buffer solution having an appropriate concentration of protein. Table 1 shows the molecular weight, pI and ionic characteristic at pH 7.0 of five model proteins used herein.

TABLE 1

| | Molecular | | Ionic character at pH |
|---|---|---|---|
| Protein | weight | pI | 7.0 |
| Ovalbumin | 43,000 | 4.80 | Anionic |
| Bovine serum albumin | 66,000 | 5.14 | Anionic |
| Human growth hormone | 22,000 | 5.27 | Anionic |
| Ribonuclease A | 13,700 | 8.64 | Cationic |
| Lysozyme | 14,300 | 9.32 | Cationic |

Detailed incorporation procedures are as follows. Five mg of dried porous microspheres were exactly weighed and transferred into a 1.5 ml polypropylene eppendorf tube. Five hundred μl of protein solution in appropriate buffer was added. Buffers with pH range of 3-8 were prepared by appropriate mixing of 10 mM citrate and 10 mM potassium phosphate, dibasic. The tubes were then placed on a rotator wheel and constantly rotated at 50 rpm in a constant temperature incubator. At a predetermined time, tubes were removed and centrifuged. Non-incorporated protein amount in the supernatant was determined by micro-BCA protein assay kit (Pierce) using each protein as a standard. Incorporated amounts of protein into porous microspheres were calculated by subtraction of non-incorporated amounts from initially added amounts. The amount of incorporated protein co-precipitated with microspheres was also directly assayed after dissolving the microspheres with 0.1 N NaOH/0.5% SDS for 2 days at 37° C.

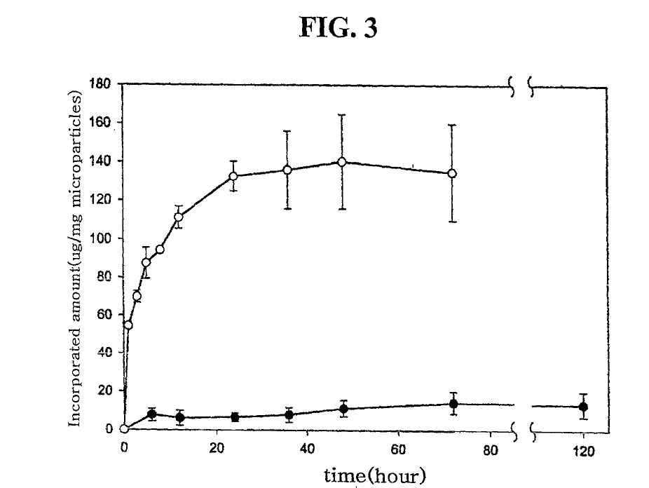

Time kinetics of lysozyme incorporation into anionic microspheres were investigated at two different temperatures, i.e., at 4° C. and 37° C. Lysozyme concentration at 2 mg/ml in pH 7.0 buffer and carboxyl group-containing microspheres of formulation-2 (made from RG502H) were used for the experiment. As shown in FIG. 3, the maximal incorporation amount at lower temperature (4° C.) was about 10% of that at higher temperature (37° C.). This may be partly due to lower degree of hydration and swelling of the microspheres at 4° C. The equilibration time of incorporation at 37° C. was determined to be about 24 hours. All the following incorporation experiments were performed at 37° C. hours.

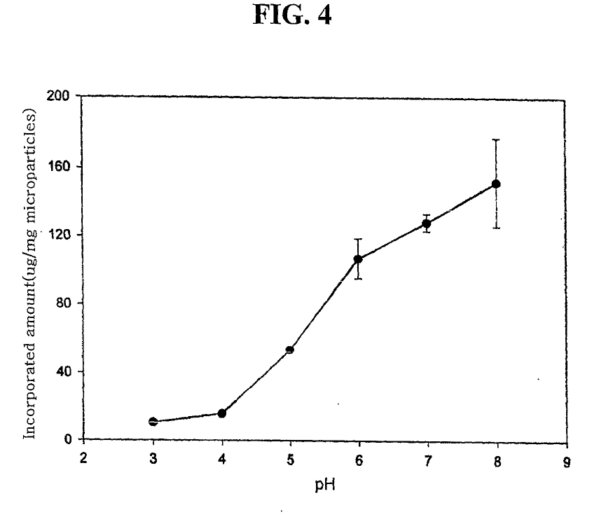

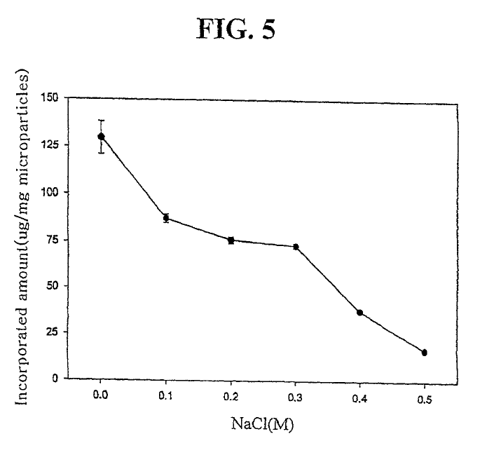

The effect of pH of the incorporation medium on the lysozyme incorporation into anionic microspheres (formulation-2) was investigated at 2 mg/ml of lysozyme concentration by varying the buffer pH from 3.0 to 8.0 and the result is shown in FIG. 4. To see the ionic strength effect on the lysozyme incorporation into anionic microspheres, NaCl concentration of the incorporation medium (pH 7.0) was varied from 0.0 to 0.5 M. FIG. 5 shows the result. These results indicate that the pH and ionic strength of the incorporation medium are critical factors of the incorporation efficiency. These results also indicate that the incorporation is mainly caused from ionic interaction because the incorporation efficiency is decreased as the pH of the incorporation medium is lowered (carboxyl group is losing its ionic characteristic) or NaCl concentration is increased. All the following incorporation experiments were performed at pH 7.0.

Effects of increase of microsphere porosity by addition of porosigens such as NaCl and ammonium bicarbonate and increase of carboxyl groups of microsphere by addition of carboxyl group-containing biocompatible excipients such as citric acid or caprylic acid, on the lysozyme incorporation were studied for anionic microspheres of formulation-1, -2, -3, -4, and -5. Table 2 shows the results.

TABLE 2

| Formulation | Composition of primary water | Lysozyme loading % |
|---|---|---|
| No. | phase | (w/w)^{a} |
| 1 | Deionized water | 9.20 |
| 2 | 0.5 M NaCl | 10.58 |
| 3 | 0.5 M citric acid | 14.11 |
| 4 | 0.5 M caprylic acid | 13.76 |
| 5 | 0.5 M ammonium bicarbonate | 10.84 |

^{a}: lysozyme loading % was calculated by weight of lysozyme incorporated/weight of lysozyme-incorporated microspheres × 100.

Lysozyme loading amount was increased by addition of either porosigen or carboxyl group-containing excipient. Particularly, the effect of the addition of carboxyl group-containing excipients such as citric acid and caprylic acid was more prominent, which demonstrates the improvement of the present invention.

The applicability of the present invention to a broad range of biopharmaceuticals is demonstrated by the results shown in Table 3. Relative incorporation efficiencies of five model proteins those have different pI values were tested toward several microsphere formulations those have different ionic characteristics. At 2 mg/ml of protein concentration was used for this experiment.

TABLE 3

| | Formulation | Protein loading % (w/w) | | | | |
|---|---|---|---|---|---|---|
| No. | Main components of microsphere | OVA | BSA | hGH | Rib A | LYS |
| 2 | RG502H | 0.75 | 1.36 | 0.99 | 10.00 | 11.84 |
| 6 | RG502 | <0.20 | <0.20 | 0.46 | 1.90 | <0.20 |
| 7 | 90% RG502, 10% benzalkonium | 3.30 | 9.41 | 16.83 | 0.81 | <0.20 |
| | Cl | | | | | |
| 11 | 95% RG502H, 5% benzalkonium | 2.51 | <0.20 | 8.22 | 7.90 | 6.03 |
| | Cl | | | | | |
| 15 | 99.9% RG502, 0.1% chitosan | 1.91 | <0.20 | 5.61 | 5.27 | 0.67 |

Into the non-ionic microspheres of formulation-6, incorporated amounts are very low for all five proteins. Cationic proteins such as ribonulease A and lysozyme were highly incorporated into anionic microspheres of formulation-2, whereas anionic proteins such as ovalbumin, bovine serum albumin, and hGH were highly incorporated into cationic microspheres of formulation-7. Into the amphoteric microspheres of formulation-11, incorporated amounts are relatively high for all proteins except for bovine serum albumin. Chitosan, glucosamine-containing cationic polysaccharide, was also found to have a potential to increase the incorporation of an anionic protein, hGH, into the microspheres. Above all the results suggest that appropriate blending of ionic polymer or ionic excipient with biodegradable polymer can make microspheres having various ionic characteristics which can be applied to a broad range of biopharmaceuticals with various degrees of incorporation capacities.

To investigate the maximal incorporation capacity of the cationic microspheres of formulation-7 for hGH, the concentration of the protein in the incorporation medium was varied from 2 mg/ml up to 20 mg/ml with the fixed he microspheres, i.e. 10 mg/ml. Table 4 shows the results.

TABLE 4

| HGH concentration | | Loading efficiency |
|---|---|---|
| (mg/ml) | hGH loading % (w/w)^{a} | (%)^{b} |
| 2 | 16.83 | 101.2 |
| 5 | 31.75 | 93.0 |
| 10 | 49.96 | 99.8 |
| 20 | 63.66 | 87.6 |

^{a}: hGH loading % was calculated by weight of hGH incorporated/weight of hGH-incorporated microspheres × 100.
^{b}: Loading efficiency was calculated by weight of hGH incorporated/weight of hGH added × 100.

As the concentration of hGH in the incorporation medium was increased, loading % was also increased resulting in a maximal loading % of 63.66 at 20 mg/ml concentration of hGH. This result further demonstrates the advance and superiority of the present invention in respect that a biopharmaceutical can be incorporated greater than 60% into biodegradable microspheres by a non-invasive method.

### EXAMPLE 3

### In Vitro Release of hGH from the Biodegradable Microspheres

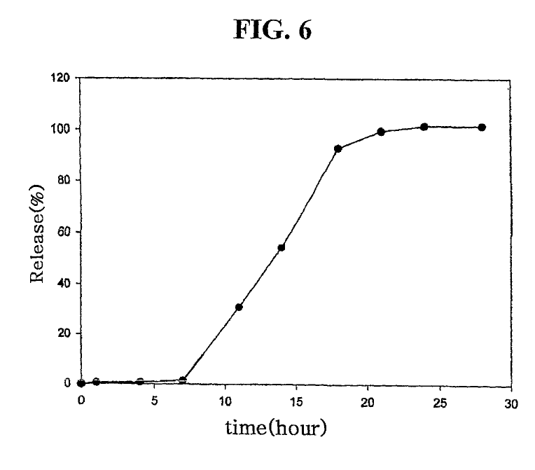

One hundred mg of hGH-loaded microspheres (hGH content (w/w): 16.83%, Formulation-7) were exactly weighed and suspended in 1 ml of 10 mM phosphate buffer (pH 7.4). The tubes were then placed in an incubator at 37° C. At predetermined time, tubes were removed and centrifuged. Released hGH amount in the supernatant was determined by micro-BCA protein assay kit (Pierce) using hGH as a standard. The precipitated microspheres were re-suspended with 1 ml of fresh buffer and incubated at 37° C. for further release experiment. Samples were collected at 1, 4, 7 days after the starting time and twice weekly thereafter for 28 days. As shown in FIG. 6, hGH was released at constant rate to complete release at 21 days after lag period during initial 7 days.

Those skilled in the art will recognize, or be able to ascertain many equivalents to specific embodiments of the present invention using no more than usual experimentation. Such equivalents are intended to be encompassed in the scope of the following claims.

What is claimed is:

1. A process to prepare an injectable sustained release pharmaceutical composition comprising a step to prepare biodegradable porous microspheres having accessible ionic functional groups, a step to incorporate a biopharmaceutical into the microspheres through ionic interaction by suspending or equilibrating the microspheres in a solution containing the biopharmaceutical and a step to recover and freeze-dry the biopharmaceutical-incorporated microspheres.

2. The process of claim l, wherein the composition is prepared by incorporation of a cationic biopharrnaceutical into biodegradable porous microspheres having anionic functional groups and wherein the pH of incorporation solution is lower than the pI of the biopharmaceutical.

3. The process of claim 1, wherein the composition is prepared by incorporation of an anionic biopharmaceutical into biodegradable porous microspheres having cationic functional groups and wherein the pH of incorporation solution is higher than the pI of the biopharmaceutical.

4. The process of claim 1-3, wherein said biopharmaceutical is present in an amount from 0.1% to 90% weight.

5. The process of claim 1-3, wherein said biodegradable polymer is one or more of polylactides, polyglycolides, poly(lactide-co-glycolide)s, polycaprolactone, polycarbonates, polyesteramides, polyanhydrides, poly(amino acids), polyolthoesters, polyacetyls, polycyanoacrylates, polyetheresters, poly(dioxanone)s, poly(alkylene alkylate)s, copolymers of polyethylene glycol and polyorthoester, biodegradable polyurethanes, proteins such as albumin, casein, collagen, fibrin, fibrinogen, gelatin, hemoglobin, transfferin, and zein, polysaccharides such as alginic acid, chitin, chitosan, chondroitin, dextrin, dextran, hyaluronic acid, heparin, keratan sulfate, starch and derivatives or blends thereof.

6. The process according to any of the claims 2, 4, 5, wherein said anionic functional groups are selected from carboxyl, sulfonyl and phosphoryl groups.

7. The process according to any of the claims 2, 4, 5, wherein said biodegradable porous microspheres having anionic functional groups are prepared from the blends of anionic surfactant and/or biocompatible materials having anionic functional group with biodegradable polymer.

8. The process of claim 7, wherein said anionic surfactant is selected from docusate sodium and sodium lauryl sulfate.

9. The process according to any of the claims 3, 4, 5, wherein said cationic functional groups are selected from primary to quaternary amine groups.

10. The process according to any of the claims 3, 4, 5, wherein said biodegradable porous microspheres having cationic functional groups are prepared from the blends of cationic surfactant or biocompatible materials having cationic functional group with biodegradable polymer.

11. The process of claim 10, wherein said cationic surfactant is selected from benzalkonium chloride, benzethonium chloride, and cetrimide.

12. The process according to any of the claims 1-3, wherein said biopharmaceutical is selected from the group consisting of growth hormones, interferons, colony stimulating factors, interleukins, macrophage activating factors, macrophage peptides, B cell factors, T cell factors, protein A, suppressive factor of allergy, suppressor factors, cytotoxic glycoprotein, immunocytotoxic agents, immunotoxins, immunotherapeutic polypeptides, lymphotoxins, tumor necrosis factors, cachectin, oncostatins, tumor inhibitory factors, transforming growth factors, albumin and its fragments, alpha-1 antitrypsin, apolipoprotein-E, erythroid potentiating factors, erythropoietin, factor VII, factor VIII, factor IX, fibrinolytic agent, hemopoietin-1, kidney plasminogen activator, tissue plasminogen activator, urokinase, prourokinase, streptokinase, lipocortin, lipomodulin, macrocortin, lung surfactant protein, protein C, protein 5, C-reactive protein, renin inhibitors, collagenase inhibitors, superoxide dismutase, epidermal growth factor, platelet derived growth factor, osteogenic growth factors, atrial naturetic factor, auriculin, atriopeptin, bone morphogenetic protein, calcitonin, calcitonin precursor, calcitonin gene-related peptide, cartilage inducing factor, connective tissue activator protein, fertility hormones (follicle stimulating hormone, leutinizing hormone, human chorionic gonadotropin), growth hormone releasing factor, osteogenic protein, insulin, proinsulin, nerve growth factor, parathyroid hormone, parathyroid hormone inhibitors, relaxin, secretin, somatomedin C, insulin-like growth factors, inhibin, adrenocorticotrophic hormone, glucagons, vasoactive intestinal polypeptide, gastric inhibitory peptide, motilin, cholecystolinin, pancreatic polypeptide, gastrin releasing peptide, corticotropin releasing factor, thyroid stimulating hormone, vaccine antigens of, and anti-infective antibodies to, bacterial or viral or other infectious organisms and mutants or analogs thereof.

13. The process according to any of the claims 1-3, wherein said biodegradable porous microspheres having ionic functional groups are prepared by a method selected from solvent extraction or evaporation in aqueous or organic phase, phase separation, spray drying, low temperature casting and supercritical gas fluid method.

14. The process according to any of the claims 1-3, wherein porosity of said biodegradable porous microspheres having ionic functional groups is intended to be increased by addition of gas forming agents or salts such as sodium chloride, calcium chloride and ammonium bicarbonate during microsphere preparation process.

15. The process according to any of the claims 1-3, wherein said biodegradable porous microspheres having ionic functional groups are prepared by co-addition of acidifying agents such as lactic acid, glycolic acid, tartaric acid, citric acid, fumaric acid, and malic acid, alkalizing agents such as diethanolamine, monoethanolamine, potassium citrate, sodium bicarbonate, calcium carbonate, magnesium carbonate, magnesium oxide, magnesium trisilicate, sodium citrate, meglumine, and triethanolamine and salts.

16. The process according to any of the claims 1-3, wherein the incorporation of a biopharmaceutical into said biodegradable porous microspheres having ionic functional groups are performed in an aqueous buffer solution, where the pH of the buffer is from 3.0 to 9.0, salt concentration of the buffer is from 1 to 500 mM, incorporation temperature is from 5 to 50° C. and incorporation time is from 1 minute to 20 days.

17. The process of the claim 16, wherein the salt concentration of the buffer is from 5 to 200 mM, incorporation temperature is from 30 to 42° C. and incorporation time is from 10 to 48 hours.

18. The process of claim 16, wherein the incorporation medium further comprises a release rate modifying additive or excipient or a cryoprotectant.

19. The process according to any of the claims 1-3, wherein the composition is further coated with one or more of gelatin, fibrin, or albumin.

20. The process according to any of the claims 1-3, wherein the size of the microspheres is within the range from 0.01 to 500 μm.

21. An injectable sustained release pharmaceutical composition by preparing the process according to any of claims 1-20.
